(19) United States
(12) Patent Application Publication
(10) Pub. No.: US 2010/0272650 A1
(43) Pub. Date: Oct. 28, 2010

(54) SEMICONDUCTOR NANOPARTICLE, AND FLUORESCENT LABELING SUBSTANCE AND MOLECULE/CELL IMAGING METHOD BY USE THEREOF

(75) Inventors: Kazuya Tsukada, Kanagawa (JP); Kumiko Nishikawa, Tokyo (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA MEDICAL & GRAPHIC, INC., Tokyo (JP)

(21) Appl. No.: 12/742,725

(22) PCT Filed: Oct. 28, 2008
(86) PCT No.: PCT/JP2008/069525

§ 371 Date: May 13, 2010

(30) Foreign Application Priority Data
Nov. 21, 2007 (JP) 2007301332

(51) Int. Cl. A61B 5/00; H01L 29/66; C12Q 1/68

(52) U.S. Cl. 424/9.6; 257/14; 435/6; 257/E29.168; 977/773

(57) ABSTRACT

There is provided semiconductor nanoparticles which exhibit enhanced emission efficiency, excellent emission intensity, reduced variation range of emission characteristics among lots and among particles and are excellent in stability and reproducibility. There is further provided a fluorescent labeling agent and molecule/cell imaging method by use of the same. Semiconductor nanoparticles having an average particle size of 1 to 20 nm is disclosed, comprising a dopant of a heteroatom which is identical in valence electron configuration with a main component atom forming the semiconductor nanoparticles or an atomic pair of the heteroatom, and the dopant is distributed on or near a surface of the semiconductor nanoparticles.

### TECHNICAL FIELD

The present invention relates to semiconductor nanoparticles, and a fluorescent labeling agent and a molecule/cell imaging method by use thereof.

### TECHNICAL BACKGROUND

There has been active fundamental research of molecular imaging with intention of revealing the molecular dynamic state, molecular interaction and molecular location information by visualization of imaging targeting molecules in a living body of a living cell or a small animal as an object and connecting them to elucidation of life science mechanism or screening of new drugs. Conventional labeling agents probing a biomolecule have generally employed fluorescent organic dyes, organic fluorescent proteins and a luciferase (enzyme)-luciferin (substrate) light-emitting body. These labeling agents have been desired to enhance detection sensitivity for fluorescence emission and specifically have not yet met the desired imaging in deeper regions in vivo of a small animal. In response to these desires, there have been made studies to form a labeling agent having a novel structure to achieve enhanced emission or fluorescence intensity.

However, when enhancing an exciting light intensity to achieve enhanced detection sensitivity, there result problems such that light toxicity brings about invasiveness to a living body molecule or a label itself is easily photolyzed and is of poor durability.

Recent advances in nanotechnology suggest the possibility of employing so-called nanoparticles for detection, diagnosis, sensitiveness or other uses. Recently, nanoparticle composite materials capable of interacting with a biological system broadly attract attention in the field of biology or medical science. Such composite materials are expected to be useful as a novel intravascular probe for both of sensitiveness (for example, imaging) or therapeutic purpose (for example, drug delivery).

It is well known that, among metal or semiconductor ultra-fine particles, nano-sized particles having a smaller particle size than an electron wavelength (approximately, 10 nm) are greatly affected by the finite nature of particle size on the motion of an electron, as a quantum effect and exhibit specific physical properties differing from its bulk body (as described in Non-patent document 1).

A substance which is composed of a nanometer-sized semiconductor material and exhibits a quantum confinement effect, for example, a semiconductor nanoparticle, is generally called a quantum dot. Such a quantum dot, which is a small agglomerate of some ten nms and composed of some hundreds to some thousands of semiconductor atoms, emits an energy equivalent to the energy band gap of the quantum dot when absorbing light from an exciting source and reaching an energy-excited state. Therefore, it is considered that controlling the size or material composition of a quantum dot can adjust the energy band gap, enabling to employ energy of a wavelength band at various levels.

Further, quantum dots, that is, semiconductor nanoparticles exhibit characteristics such that the emission wavelength can be controlled by varying the particle size in an identical composition. It is also noted that they are superior in stability and emission luminance, compared to organic fluorescent dyes as in the prior art.

It is also known that when such quantum dots are minimized to a particle size exhibiting a quantum effect, enhanced emission efficiency is achieved by a quantum confinement effect which brings about strong excitons, whereby visually observable emission is obtained.

However, such fluorescence emission is insufficient to be used as various detecting agents, leading to various kinds of efforts to achieve enhanced luminance. For instance, there is known a means in which a shell is formed on the surface of a silicon semiconductor nanoparticle (quantum dot) to decrease defects on the particle surface to passivate the surface, thereby achieving enhanced emission efficiency. However, such a means known in the prior art is insufficient to obtain high emission and is also insufficient in stability and reproducibility between lots, requiring improvements thereof.

It is a fact that emission of a silicon nanoparticles (quantum dots), which is emitted by indirect transition via a phonon and is extremely low in emission intensity, is often insufficient for silicon which emits almost no emission at a bulk particle diameter. There was disclosed a technique of employing isoelectron traps to achieved enhanced emission without being affected by indirect transition (as described in, for example, lecture preprint 6a-L-4 of 68th Meeting of Oyobutsuri-Gakkai), however, such a technique was insufficient in stability, producing problems such that even in an identical lot, emission differs between particles, resulting in instability in emission.

Research and development in the technical field related to the foregoing semiconductor nanoparticles (quantum dot), specifically, research and development aiming at application in the field of biology and medical science have begun and in the situation of problems remaining to be solved (as set forth in, for example, Patent documents 1-3).

Patent document 1: JP 2004099349A

Patent document 2: JP 2005-314408A

Patent document 3: JP 2005-101601A

Non-patent document 1: “Nikkei Sentan Gijutsu” No. 27, pp 1-4, January 2003.

### DISCLOSURE OF THE INVENTION

### Problem to be Solved

The present invention has come into being in view of the forgoing problems and circumstances. One problem to be solved is to provide semiconductor nanoparticles which exhibit enhanced emission efficiency, excellent emission intensity, reduced variation range of emission characteristics among lots and among particles and are excellent in stability and reproducibility. It is further to provide a fluorescent labeling agent and molecule/cell imaging method by use of the same.

As a result of extensive study by the inventors of this application to solve the foregoing problems, it was found that fluorescence emission of high stability and enhanced emission intensity was achieved by allowing atoms which are identical in valence electron configuration with a main constituent atom constituting a semiconductor nanoparticle parent body to be uniformly distributed on a surface or near the surface, whereby the invention was achieved.

The foregoing problems related to the invention was solved by the following constitution:

1. Semiconductor nanoparticles having an average particle size of 1 to 20 nm, comprising a dopant of a heteroatom which is identical in valence electron configuration with a main component atom forming the semiconductor nanoparticles or an atomic pair of the heteroatom, and the dopant being distributed on or near the surface of the semiconductor nanoparticles.

2. The semiconductor nanoparticles as described in 1, wherein the main component atom is silicon (Si) or germanium (Ge).

3. The semiconductor nanoparticles as described in 1 or 2, wherein the atomic pair is Be—Be.

4. The semiconductor nanoparticles as described in any of 1 to 3, wherein the dopant is distributed within a region of from the surface of the semiconductor nanoparticles to 30% of a radius of the semiconductor nanoparticles.

5. A fluorescent labeling substance, wherein a surface-modifying compound which is affinitive for or connective to a living body is disposed on the surface of nanoparticles described in any of the foregoing 1 to 4.

6. A molecular and cellular imaging method of visualizing a molecule within a targeted cell through fluorescence emitted by a fluorescent labeling substance, wherein the fluorescent labeling substance contains semiconductor nanoparticles, as described in any of the foregoing 1 to 4.

### EFFECT OF THE INVENTION

According to the foregoing means, there can be provided semiconductor nanoparticles which exhibit enhanced emission efficiency, excellent emission intensity, reduced variation range of emission characteristics among lots and among particles and are excellent in stability and reproducibility. Further, there can also be provided a fluorescent labeling agent and molecule/cell imaging method by use thereof.

The foregoing effects of the invention were based on the results that enhanced emission efficiency was achieved by doping an atom which is identical in valence electron configuration with a main constituent atom constituting a semiconductor nanoparticle parent body and specifically, fluorescence emission, not caused by indirect transition, was obtained by silicon (Si) semiconductor nanoparticles, resulting in enhanced emission efficiency. Further, no influence of surface defects was caused by allowing a dopant to be localized on the surface or in the vicinity thereof, whereby enhanced emission efficiency and minimized variation range of emission characteristics among lots and among particles were attained, resulting in enhanced stability and reproducibility.

### PREFERRED EMBODIMENTS OF THE INVENTION

The semiconductor nanoparticles of the invention are featured in that the semiconductor nanoparticles exhibit an average particle size of 1 to 20 nm and contain a dopant of a heteroatom which has the same valence electron configuration as a main component atom constituting the nanoparticles or an atomic pair of the heteroatom, and the dopant is distributed on or near the surface of the semiconductor nanoparticles. This feature is a technical feature in common with the foregoing items 1-6 of the invention.

In the embodiments of the invention, the main component atom preferably is silicon (Si) or germanium (Ge), and the atomic pair preferably is Be—Be.

Further, it is preferred that the dopant is distributed within the range of from the surface to 30% of the radius of the semiconductor nanoparticles.

The semiconductor nanoparticles of the invention is applicable to a fluorescent labeling substance by allowing a surface-modifying compound which is affinitive with or capable of connecting to a living body to be disposed on the surface of nanoparticles

The fluorescent labeling substance is appropriately applicable to a molecule/cell imaging method of visualizing a molecule within a targeted cell through fluorescence emitted by the fluorescent labeling substance.

In the following, there will be described constituent elements of the invention and preferred embodiments of the invention.

### Semiconductor Nanoparticle

Materials used for the semiconductor nanoparticles related to the invention may employ various fluorescence-emitting compounds known in the art and raw materials for them. For instance, various semiconductor material which have been known as a material used for semiconductor nanoparticles may be employed as a raw material. Specifically there may be employed, for example, semiconductor compounds of group IV, group and group of the periodic table and raw material compounds containing elements constituting the semiconductor materials.

Examples of a group II-VI semiconductor include MgS, MgSe, MgTe, CaS, CaSe, CaTe, SrS, SrSe, SrTe, BaS, BaSe, BaTe, ZnS, ZnSe, ZnTe, CdS, CdSe, HgS<HgSe and HgTe.

Examples of a group semiconductor include GaSe, GaN, GaP, GaSb, InP, InN, InSb, InAs, AlAs, AlP, AlSb and AlS.

Among group IV semiconductors, Ge and Si are specifically suitable. Among the foregoing semiconductor materials, Si, Ge, InN and InP are specifically preferred in terms of composition meeting safety and further of these, Silicone (Si) and germanium (Ge) are specifically preferred as a main component atom forming the semiconductor nanoparticles of the invention. In the invention, the expression, “the main component atom forming the semiconductor nanoparticles” refers to an atom exhibiting the maximum content among atoms forming the semiconductor nanoparticles.

In the present invention, preferably, semiconductor phosphor nanoparticles have a core/shell structure. In such a case, it is preferred that semiconductor phosphor nanoparticles are those which have a core/shell structure constituted of a core particle of a semiconductor particle and a shell layer covering the core particle, and that the core particle differs in chemical composition from the shell layer. Accordingly, it is preferred that the band gap of the shell is higher than that of the core.

A shell is necessary to stabilize surface defects and enhance luminance and is also important to form the surface onto which a surface-modifying agent easily adsorbs. It is also an important constitution to achieve enhanced precision of the detection sensitivity for the effect of the invention.

There will be described a core particle and a shell layer.

### Core Particle

Semiconductor materials used for core particles may employ a various kinds of semiconductor materials. Specific examples thereof include MgS, MgSe, MgTe, CaS, CaSe, CaTe, SrS, SrSe, SrTe, BaS, BaTe, ZnS, ZnSe, ZnTe, CdS, CdSe, CdTe, GaAs, GaP, GaSb, InGaAs, InP, InN, InSb, InAs, AlAs, AlP, AlSb, AlS, PbS, PbSe, Ge, Si and a mixture of these. In the invention, a specifically preferred semiconductor material is Si.

The average particle size of the core related to the invention is preferably from 0.5 to 15 nm.

In the invention, the average particle size of semiconductor phosphor nanoparticles needs to be determined three-dimensionally but it is difficult to determine the particle size in such a manner because of its being extremely minute. Actually, it has to be determined in a two-dimensional image, so that it is preferred to determine an average size in such a manner that electronmicrographs are taken using a transmission electron microscope (TEM) to perform averaging. Thus, electronmicrographs are taken using a TEM and a sufficient number of particles are measured with respect to cross-sectional area to determine the diameter of a circle, equivalent to the cross-sectional area and an arithmetic average thereof is defined as the average particle size. The number of particles to be photographed by a TEM is preferably at least 100 particles.

In the semiconductor nanoparticles related to the invention, the average core particle size is preferably controlled so that the nanoparticles emit a fluorescence at the wavelength in the infrared region, that is, infrared-emit.

### Shell Layer

Semiconductor materials used for a shell may employ various kinds of semiconductor materials. Specific examples thereof include SiO₂, ZnO, ZnS, ZnSe, ZnTe, CdO, CdS, CdSe, CsTe, MgS, MgSe, GaS, GaN, GaP, GaAs, GaSb, InAs, InN, InP, InSb, AlAs, AlN, AlP, AlSb and further mixtures of these.

In the invention, the specifically preferred semiconductor material is SiO₂ or ZnS.

The shell layer related to the invention need not completely cover all of the surface of a core particle unless partial exposure of the core particle has an adverse effect.

### Dopant

The semiconductor nanoparticles of the invention contain a heteroatom or an atomic pair of the heteroatom, as a dopant, and such a heteroatom is identical in valence electron configuration with a main component atom forming the semiconductor nanoparticles and the dopant is uniformly distributed on or near the surface of the semiconductor nanoparticles.

Herein, “valence electron” refers to an electron which belongs to the outermost shell of electron shells (K shell, L shell, M shell, etc) constituting an atom. Therefore, in cases when the main component atom forming the semiconductor nanoparticles is silicon (Si), the valence electron is of four electrons in the outermost shell and an atom or an atomic pair having an equivalent valence electron configuration includes, for example, Be—Be (a Be pair), Mg—Mg (a Mg pair) and Ge.

In cases when the component atom forming the semiconductor nanoparticles of the invention is silicon (Si) or germanium (Ge), the dopant is preferably Be—Be.

In the invention, the location in which a dopant is contained is required to be on the surface of the semiconductor nanoparticles or near the surface of the semiconductor nanoparticles. Herein, “near the surface” is the region from the surface of the semiconductor nanoparticles to 30% of the radius of the nanoparticles, and preferably 15%.

The distribution of dopants can be observed or measured by X-ray photoelectron spectrometry (XPS/ESCA; XPS: X-ray Photoelectron Spectroscopy/ESCA: Electron Spectroscopy for Chemical Analysis). The X-ray photoelectron spectrometry is a method to investigate the state (for example, element composition) of the solid surface or in the vicinity thereof by measuring the kinetic energy of an electron ejected upon exposure to monochromatic light (X-ray).

### Particle Size of Semiconductor Nanoparticle

The average particles size of the semiconductor nanoparticles related to the invention is preferably from 1 to 20 nm and more preferably from 1 to 10 nm.

It is well-known that, of semiconductor nanoparticles related to the invention, in nano-sized particles having a smaller particle size than the electron wavelength (approximately, 10 nm), in which the influence of finiteness of size on the motion of electrons, as the quantum size effect becomes larger, exhibit a specific physical property differing from the bulk body. In general, semiconductor nanoparticles which are a nanometer-sized semiconductor substance and exhibit a quantum confinement effect are also called “quantum dot”. Such a quantum dot is a minute mass within ten and some nm, collected of some hundreds to some thousands semiconductor atoms and liberates an energy corresponding to the energy band gap of the quantum dot when it reaches an energy-excited state on absorption of light from an excitation source. Accordingly, control of the energy band gap can be achieved by controlling the size or material composition of a quantum dot, whereby energy of wavelength bands at various levels can be employed. Further, a quantum dot, that is, semiconductor nanoparticles are featured in that the emission wavelength can be controlled by variation of particle size on the same composition.

Semiconductor nanoparticles related to the invention can be controlled so as to exhibit fluorescence in the range of 350 to 1100 nm but in the invention, to minimize effects of the emission of a living body cell and achieve enhanced SN ratio, an emission of a wavelength in a near-infrared region is preferably used.

### Production Method of Semiconductor Nanoparticles

Semiconductor nanoparticles related to the invention can be produced by a liquid phase process or gas phase process known in the art.

Production methods by a liquid phase process include, for example, a coprecipitation method, a sol-gel method, a homogeneous precipitation method and a reduction method. There are further included methods superior in production of nanoparticles, such as a reverse micelle method and a supercritical hydrothermal synthesis method (as described in, for example, JP 2002-322468A, JP 2005-239775A, JP 10-310770A, and JP 2000-104058A).

A producing method of an assembly of semiconductor phosphor nanoparticles is preferably a method comprising a step of reducing a semiconductor material precursor through reduction reaction. Further, in one preferred embodiment of the invention, the reaction of such a semiconductor material precursor is performed in the presence of a surfactant. A semiconductor material precursor related to the invention is a compound containing an element used for the above-described semiconductor material and, for example, in the case of the semiconductor material being Si, SiCl₄ is cited as a semiconductor material precursor. Other examples of a semiconductor material include InCl₃, P(SiMe₃)₃, ZnMe₂, CdMe₂, GeCl₄ and tributylphosphine selenium.

The reaction temperature is not specifically limited if it is not less than the boiling point of the semiconductor material precursor and not more than the boiling point of the solvent, but is preferably in the range of 70 to 110° C.

### Reducing Agent

A reducing agent used for reduction of a semiconductor material precursor can be chosen from a variety of reducing agents known in the art, in accordance with reaction conditions. In the invention, reducing agents such as lithium aluminum hydride (LiAlH₄), sodium borohydride (NaBH₄), sodium aluminum bis(2-methoxyethoxy)hydride, lithium tri(sec-butyl)borohydride [LiBH(sec-C₄H₉)₃], potassium tri(sec-butyl)borohydride and lithium triethylborohydride are preferred in terms of reducing strength. Of these, lithium aluminum hydride (LiAlH₄) is specifically preferred in terms of reducing strength.

### Solvent

A variety of solvents known in the art are usable as a solvent to disperse a semiconductor material precursor. Preferred examples thereof include alcohols such as ethyl alcohol, sec-butyl alcohol and t-butyl alcohol; and hydrocarbon solvents such as toluene, decane and hexane. A hydrophobic solvent such as toluene is specifically preferred as a solvent for use in these dispersion.

### Surfactant

There are usable a variety of surfactants known in the art in the invention, including anionic, non-ionic, cationic, and amphoteric surfactants. Of these are preferred quaternary ammonium salts, such as tetrabutylammonium chloride, bromide, or hexafluorophosphate; tetraoctylammonium bromide (TOAB), and tributylhexadecylphosphonium bromide.

A reaction by a liquid phase process is greatly variable according to the state of a compound in liquid including a solvent. There is required attention specifically when producing nano-sized particles superior in mono-dispersibility. In a reverse micelle method, for example, the size or state of reversed micelles which forms a reaction field is varied by the concentration or kind of a surfactant used therein, so that the condition to form nanoparticles is restricted. Accordingly, an appropriate surfactant is required to be combined with a solvent.

Production methods by a gas phase process include (1) a method in which a raw material semiconductor is evaporated by a first high temperature plasma generated between opposed electrodes and allowed to pass through a second high temperature plasma generated through electrodeless discharge in a reduced pressure environment (as described in, for example, JP 6-279015A), (2) a method in which nanoparticles are separated from an anode composed of a raw semiconductor material through electrochemical etching (described in, for example, JP 2003-515459A, (3) a laser ablation method (described in, for example, P 2004-356163A), and (4) a high-speed sputtering method (described in, for example, JP 2004-296781A). There is also preferably employed a method in which a raw material gas is subjected to a gas phase reaction in a low pressure state to synthesize a powder containing particles.

Post-Treatment after Formation of Semiconductor Nanoparticle

In the production method of semiconductor nanoparticles, it is preferred that any one of post-treatment by plasma, heating, radiation or ultrasonic waves is included after formation of semiconductor nanoparticles, specifically after shell formation.

An appropriate plasma treatment may be chosen from low temperature/high temperature plasma, microwave plasma and atmospheric plasma, of which the microwave plasma is preferred.

A heat treatment can be chosen among atmosphere, vacuum and inert gas regions and applied heating, and the applied temperature range differs, depending on the constitution of phosphor particles. An excessively high temperature often causes strain or flaking between the core and the shell. A low temperature results in poor effect and a range of 100 to 300° C. is preferably employed.

A radiation treatment employs high-energy X-rays, γ-rays or neutron rays, or low-energy vacuum ultraviolet (UV) rays, ultraviolet rays or short-pulse laser rays. Treatment time depends on the kind of a radiation. For instance, X-rays, which exhibit high penetrability, often perform exposure within a relatively short time; on the contrary, ultraviolet rays require exposure over a relatively longtime.

Effects of these post-treatments are not elucidated in principle but it is assumed that adhesiveness at the interface between core and shell is reinforced and passivation is accelerated, resulting in enhanced emission efficiency. It is also assumed that such an influence is remarkable in an infrared emitter and is reflected in its characteristics.

In the invention, the band gap of a shell is preferably higher than that of its core. A shell is needed to stabilize surface defects on the core particle surface and to achieve enhanced illuminance, and is also important to form a surface onto which a surface-modifying agent is easily adhered, when used as a fluorescent labeling agent.

### Fluorescent Labeling Substance

The semiconductor nanoparticles of the invention, of which the surface is provided with an appropriate surface-modifying agent, is applicable to a fluorescent labeling substance (or a fluorescent labeling agent) to fluorescence-label a targeted substance (or a target). Specifically, a surface-modifying compound which is affinitive to or connective to a living body is disposed on the particle surface, which is suitably used as a biomolecule fluorescence labeling agent (biosubstance fluorescence labeling agent) to fluorescence-label a targeted substance such as a protein or a peptide.

When used as a biomolecule fluorescence labeling agent (biosubstance fluorescence labeling agent), it is preferred in terms of non-invasiveness and penetrability for living tissue to control an emission characteristic through particle size, or the like so that infrared light is emitted by excitation of near-infrared to infrared.

In the invention, a surface-modifying compound preferably is one which contains at least one functional group and at least one group capable of bonding to a semiconductor nanoparticle. The latter is a hydrophobic group capable of adsorbing to a hydrophobic semiconductor nanoparticle and the former is a functional group which is affinitive with a living substance and capable of bonding a biomolecule. Surface-modifying compounds may use a linker which allows them to be combined with each other.

A group capable of bonding to a semiconductor nanoparticle may be any functional group capable of bonding to a semiconductor material to form semiconductor nanoparticles. In the invention, such a functional group preferably is a mercapto group (or a thiol group).

Examples of a functional group capable of affinity-bonding to a biosubstance include a carboxy group, an amino group, a phosphonic acid group and a sulfonic acid group.

Herein, the biosubstance refers to a cell, DNA, RNA, oligonucleotide, protein, antigen, antibody, endoplasmic reticulum, nuclear, a Golgi body and the like.

To be allowed to bond to semiconductor nanoparticles, a mercapto group may be allowed to bond by adjusting the pH to a value suitable for surface modification. To the other end is introduced an aldehyde group, an amino group or a carboxyl group to than a peptide bonding with an amino group or a carboxyl group. Introduction of an amino group, an aldehyde group or a carboxyl group to DNA, oligonucleotide or the like can similarly form a bond.

Specific examples of a method of preparing a biomolecule fluorescent labeling agent (biosubstance fluorescent labeling agent) include a method in which hydrophilized semiconductor nanoparticles are linked to a molecule labeling substance via an organic molecule. In a biomolecule fluorescent labeling agent (biosubstance fluorescent labeling agent) prepared by this method, a molecular labeling substance specifically bonds to and/or reacts with a targeted a biosubstance, making it feasible to perform fluorescence labeling of the biosubstance.

Examples of the molecule labeling substance include a nucleotide chain, antigen, antibody, and cyclodextrin.

Any organic molecule, which is capable of linking a semiconductor nanoparticle and a molecular labeling agent, is not specifically limited and, for example, among proteins, albumin, myoglobin or casein, or biotin together with avidin is preferable. The binding mode is not specifically limited, including a covalent bond, ionic bond, hydrogen bond, coordination bond, physical adsorption and chemical adsorption. Of these, a bonding with high bonding strength, such as a covalent bond is preferred in terms of bonding stability.

Specifically, in the case of semiconductor phosphor nanoparticles being hydrophilized with mercaptoundecanoic acid, avidin is used together with biotin. In that case, carboxyl groups of the hydrophilized nanoparticles are appropriately covalent-bonded to avidin, further, this avidin is selectively bonded to biotin and this biotin is bonded to a biomaterial labeling agent to form a biomaterial labeling agent.

### Hydrophilization of Semiconductor Nanoparticle

The particle surface of the foregoing semiconductor nanoparticle assembly is generally hydrophobic. For example, in cases when used as a biomaterial labeling agent, the particles are poorly dispersed in water as they are, producing problems such as coagulation. Accordingly, it is preferred to subject the surface of semiconductor phosphor nanoparticles to a hydrophobilization treatment.

Such a hydrophobilization treatment is conducted, for example, in such a manner that after removal of hydrophobic substances with pyridine or the like, a surface-modifier is chemically or physically bound to the particle surface. A preferred surface-modifier is one containing a carboxyl or amino group as a hydrophilic group. Specific examples of such a surface-modifier include mercaptopropionic acid, mercaptoundecanoic acid and aminopropane-thiol. Specifically, for example, 10⁻⁵ g of core/shell type Ge/GeO₂ nanoparticles are dispersed in 10 ml pure of water containing 0.2 g of mercaptoundecanoic acid and stirred at 40° C. for 10 min. to subject the shell surface to the treatment, whereby the shell surfaces of the nanoparticles are modified with a carboxyl group.

Specific preparation for surface modification of semiconductor nanoparticles may be conducted in accordance with methods, as described in, for example, Dabbousi et al., J. Phys. Chem. B101 (1997); Hines et al., J. Phys. Chem. 100: 468-471 (1996); Peng et al., J. Am. Chem. Soc. 119, 7019-7029 (1997); and Kuno et al., J. Phys. Chem. 106: 9869 (1997).

### Biomolecule Detection System by Use of Fluorescent Labeling Substance

The fluorescent labeling substance related to the invention, having the foregoing characteristic, is suitably applicable to a biomolecule detection system, feature in that the fluorescent labeling substance is supplied to a living cell or a living tissue and fluorescence emitted by exciting semiconductor nanoparticles with radiation is detected, whereby a biomolecule in the targeted living cell or a living tissue is detected.

To a living cell or living body having a targeted (or traced) biomolecule is added a fluorescent labeling substance according to the invention and is bound or adsorbed onto the targeted material; such a bound or adsorbed material is exposed to an exciting light of a prescribed wavelength and a fluorescence at a specific wavelength, which is emitted from semiconductor phosphor particles, is detected to perform fluorescent dynamic imaging of the targeted (or traced) material. Thus, a fluorescent labeling substance related to the invention can be employed for a bio-imaging method (technical means to visualize a bio-molecule constituting a biomaterial or its dynamic phenomenon).

Examples of radiation used for excitation include visible light of a halogen lamp or a tungsten lamp, an LED, a near-infrared laser light, an infrared laser light, X-rays, and γ-rays.

### Molecular and Cellular Imaging Method

The semiconductor nanoparticles of the invention is usable as a fluorescent labeling substance by allowing a probe molecule (molecule for searching) to be bound to a molecule existing in the interior or on the surface of cell tissue as a target.

In this application, “target” refers to a biomolecule targeted by semiconductor nanoparticles, which is, for example, a protein expressed preferentially in a tissue or a cell or a Golgi body, nucleus or membrane protein. Examples of an appropriate targeted material include enzymes, proteins, cell surface acceptors, nuclear acids, lipids and phospholipids, but are not limited to these.

In the invention, it is preferred to adopt an appropriate probe molecule corresponding to a targeted (measured) substance with the purpose of imaging of the interior of a living body, dynamic measurement of a substance within a cell or the like.

A biomolecule fluorescent labeling agent (biosubstance fluorescent labeling agent) employing semiconductor nanoparticles of the invention is applicable to various molecule-cell imaging methods known in the art. Examples thereof include molecule-cell imaging methods by a laser injection method, a microinjection method, an electroporation method or the like. Of these methods is preferred application to a molecule-cell imaging method by the laser injection method.

“Laser injection method” refers to an optical method in which a laser light is irradiated directly to a cell to bore a minute hole to introduce an external substance such as a gene therethrough.

The microinjection method refers to a method in which an external substance such as a gene therethrough is mechanically introduced by air pressure using a minute needle (micropipette, microsyringe).

“Electroporation method” refers to a method in which electrical stimulation is applied to a cell to induce deformation of the cell to introduce an external substance such as a gene. For instance, employing an extracellular solution being introduced through a small pore formed in the cell membrane for a short period when a high voltage of some thousands V/cm is applies to a cell suspension at a pulse of some tens of microseconds, then a sample which is intended to be introduced, such as DNA is added to the extracellular solution and introduced into the cell.

### EXAMPLES

The present invention is described with reference to examples, but the invention is not limited to these.

### Example 1

### Preparation of Semiconductor Nanoparticles

### Quantum Dot

### Preparation of Silicone (Si) Semiconductor Nanoparticle and Be-Pair Dope Particles:

Argon gas was introduced into a vacuum chamber, then, ionized argon ions which were ionized by a high-frequency controller were allowed to collide with a target material composed of Si-tip/Be-tip/quartz glass and atoms and molecules sputterred therefrom were deposited on a semiconductor substrate to form a Be molecule-doped thin film composed of a mixture of a silicon atom and an oxygen atom.

The thus formed thin film was rapidly heated to 1100° C. in an argon environment and a heating treatment was conducted for a period necessary to allow Si to be aggregated and crystallized, whereby silicon semiconductor nanoparticles (crystals) were deposited within the film. Further, silicon (Si) semiconductor nanoparticles differing in size were also deposited by control of the annealing time. The localization position of a Be-pair (Be—Be) as a dope atom in the interior of the silicon (Si) nanoparticle was controlled by proportion of a Be chip, annealing time, temperature-increasing or-decreasing rate, and annealing temperature.

The thin film containing silicon (Si) semiconductor nanoparticles were treated with an aqueous 1% hydrofluoric acid solution at room temperature to remove the SiO₂ membrane, whereby silicon (Si) semiconductor nanoparticles were exposed. The thus exposed substrate was immersed in butanol and exposed to ultrasonicwaves to detach the silicon (Si) semiconductor nanoparticles from the substrate to obtain a dispersion of the silicon (Si) semiconductor nanoparticles. A daggling bond (unpaired bond) of the silicon (Si) atom on the semiconductor particle (crystal) surface is hydrogen-terminated by the foregoing hydrofluoric acid treatment, whereby the silicon (Si) crystal is effectively stabilized. Thus obtained silicon (Si) particles are subjected to a separation treatment through high performance liquid chromatography (HPLC) to perform complete removal of any remaining HF and by-products differing in size or constituting ratio. The average particle size and distribution were measured by using ZETA SIZER, produced by Sysmex Co., Ltd. Measurement results are shown in Table 1.

Silicon (Si) semiconductor nanoparticles differing in doping position were prepared similarly to the foregoing preparation method, provided that conditions were varied by a means to control introduction and distribution of dope atoms. In Table 1 are also shown emission intensifies of various silicon semiconductor particles when exposed to light at an exciting wavelength of 365 nm

### Evaluation of Between-Lot Stability

The respective particle preparation was conducted 50 times in the same manner as above and evaluated with respect to variation (standard deviation/average value) of emission intensity or emission wavelength (λmax), as shown in Table 1.

Distribution of Be-Pair within Particle

Using XPS, Be was detected with respect to presence ratio from the peak intensity corresponding to the depth from the surface.

### Preparation of Fluorescent Labeling Substance

Introduction of Surface Modifying Compound into Semiconductor Nanoparticles:

When labeling a biosubstance with the foregoing semiconductor nanoparticles, it is necessary to introduce a functional group or the like which is capable of bonding both the particles and the biosubstance, which is conducted as follows.

Employing bonding of mercapto groups (SH groups), a carboxy group is introduced to semiconductor nanoparticles. First, the foregoing silicon semiconductor nanoparticles were dispersed in aqueous 30% hydrogen peroxide over 10 min. to hydroxidize Si—H on the crystal surface. Then, the solvent was replaced by toluene and mercaptopropyltriethoxysilane was added in an amount of 2% to the toluene to perform introduction of a mercapto group together with formation of a silane on the outermost surface of the silicon nanoparticles over 2 hrs. Subsequently, the solvent was replaced by pure water, a buffer salt was added thereto and then, 3-mercaptopropionic acid with an attached mercapto group on one end was added in an optimal amount and stirred for 3 hrs. to allow the surface-modifying compound to be bonded to the silicon (Si) core particle surface. There was thus obtained a labeling agent A.

Using plural columns capable of selective-absorbing the individual raw material components used for preparation of the obtained labeling agent A, and being provided with size-selectivity (in which plural columns varied by the combination of the surface composition of a column and a pore diameter were used; the surface composition was a carboxy group or an amino group, the size-selectivity was fitted to the particle size of a surface-modifying material and the individual raw material components were each selectively adsorbed by lessening the pore size), the HPLC treatment in all of columns was continuously or separately performed to remove raw materials and solvents other than the labeling agent A.

### Observation of Fluoresce-Labeled Biomolecule:

The thus obtained labeling materials were each added to Vero cell culture solutions, cultured at 37° C. for 2 hrs. Thereafter, they were subjected to trypsinization, dispersed in a DMEM culture medium and then sowed into a glass bottom dish. The cell cultured at 37° C. overnight was solidified with a 4% formalin solution and the localization state of the labeled material within a cell, introduced by an end site system, was evaluated by fluorescence intensity. Observation results are shown Table 1.

TABLE 1

| | | | | | Stability | | | |
|---|---|---|---|---|---|---|---|---|
| | Particle | | | Emission | Variation in | Variation in | | |
| Sample | Size | Presence | | Intensity | Emission | Wavelength | | |
| No. | (nm) | of Dopant | Distribution/Position of Dopant*¹ | (*2) | Intensity | λmax | Cell Imaging Observation | Remark |
| 1 | 3.5 | No | — | 100 | 30% | 27% | Cell shape being observed but | Comp. |
| | | | | | | | blurred | |
| 2 | 3.5 | Yes | Being distributed overall within | 105 | 25% | 22% | Cell shape being observed but | Comp. |
| | | | quantum dot particle | | | | blurred | |
| 3 | 3.5 | Yes | Being distributed within 30% but | 108 | 25% | 20% | Cell shape being observed but | Comp. |
| | | | concentrated in a partial portion | | | | blurred | |
| 4 | 3.5 | Yes | Be pair being uniformly distributed | 140 | 13% | 10% | Cell being clearly observed | Inv. |
| | | | with 30% from the surface | | | | | |
| 5 | 3.5 | Yes | Be pair being uniformly distributed | 155 | 10% | 6% | Cell being clearly observed | Inv. |
| | | | with 15% from the surface | | | | | |

As shown in Table 1, the semiconductor nanoparticles of the invention exhibited highly enhanced emission intensity and enhanced stability. It was also proved that enhanced detection capability of a biomolecule was achieved as a labeling material.

From the foregoing results, it was shown that according to the means of the invention, there were provided semiconductor nanoparticles which exhibited enhanced emission efficiency, excellent emission intensity, reduced variation range of emission characteristics among lots and among particles and are excellent in stability and reproducibility. There were also provided a fluorescent labeling agent and a molecular and cellular imaging method by use of the same.

1. Semiconductor nanoparticles having an average particle size of 1 to 20 nm, comprising a dopant of a heteroatom which is identical in valence electron configuration with a main component atom forming the semiconductor nanoparticles or an atomic pair of the heteroatom, and the dopant is distributed on or near a surface of the semiconductor nanoparticles.

2. The semiconductor nanoparticles as claimed in claim 1, wherein the main component atom is silicon (Si) or germanium (Ge).

3. The semiconductor nanoparticles as claimed in claim 1, wherein the atomic pair is Be—Be.

4. The semiconductor nanoparticles as claimed in claim 1, wherein the dopant is distributed within a region of from the surface of the semiconductor nanoparticles to 30% of a radius of the semiconductor nanoparticles.

5. A fluorescent labeling substance, wherein a surface-modifying compound which is affinitive for or connective to a living body is disposed on the surface of nanoparticles as claimed in claim 1.

6. A molecular and cellular imaging method of visualizing a molecule within a targeted cell through fluorescence emitted by a fluorescent labeling substance, wherein the fluorescent labeling substance contains semiconductor nanoparticles, as claimed in claim 1.
